(19) United States
(12) Patent Application Publication
(10) Pub. No.: US 2002/0137216 A1
(43) Pub. Date: Sep. 26, 2002

(54) Determination of effect of ingredients and levels thereof on characteristics of dough and batter-based products

(76) Inventors: Richard Y. Chen, Manhattan, KS (US); James J. Psotka, Manhattan, KS (US); Maureen C. Olewnik, Manhattan, KS (US); Virgil W. Smail, Manhattan, KS (US); Bradford W. Seabourn, Manhattan, KS (US); Okkyung Kim Chung, Manhattan, KS (US)

Correspondence Address: HOVEY WILLIAMS LLP, Suite 400, 2405 Grand, Kansas City, MO, 64108, US

(21) Appl. No.: 10/078,084

(22) Filed: Feb. 15, 2002

Related U.S. Application Data
(63) Continuation of application No. 09/426,977, filed on Oct. 26, 1999, now abandoned.

(51) Int. Cl. G01N 33/10

(52) U.S. Cl. 436/20; 436/171

(57) ABSTRACT

A novel method of analyzing wheat-based dough and batter products is provided. The preferred method includes mixing doughs or batters having different ingredients or levels thereof, performing a series of near-infrared analyses on such doughs and batters and comparing the analyses to indicate the effect of the added ingredient or levels thereof. The spectral data collected from the analyses are converted to Kubelka-Munk units and the second derivatives are determined and the resulting time plot curve is smoothed and cumulated resulting in a development plot which is easy to read and interpret, thereby aiding in determining the effect of each tested ingredient and levels thereof The present invention also provides a method for determining ingredient effects at specific wavelengths for specific ingredients. Finally, an apparatus for use with the novel methods is also provided wherein the apparatus includes a mixing container having a window through which NIR spectroscopy can be performed on the contents in the container.

### RELATED APPLICATIONS

This is a continuation of application Ser. No. 09/426,977, filed Oct. 26, 1999.

### BACKGROUND OF THE INVENTION

1. Field of The Invention

The present invention relates to the field of dough and batter manufacture. More particularly, the invention is concerned with determining the effects of the ingredients and levels thereof on characteristics of dough and batter-based products during their manufacture. In practice, near-infrared (NIR) spectroscopy is used during dough or batter formulation to determine the effects of the ingredients and levels thereof on the ultimate characteristics of final dough and batter bakery products. Data collected during NIR spectroscopy is processed using a novel method of data processing permitting easier identification of dough differences and dough similarities resulting from differences in ingredients and levels thereof during dough processing.

2. Description of The Prior Art

Doughs and batters are complex homogeneous masses of diverse ingredients which include, at the least, flour and water. Doughs and batters are different in that doughs, which have a lower water content than batters, comprise a viscoelastic mass while batters, which have a higher water content than doughs, comprise a mass of material that is flowable and pourable. Water and flour are major components which determine dough and batter characteristics. Water interacts with gluten which is part of the protein portion of flour. The amount of water used in a dough or batter formulation and its interaction with gluten exerts a major impact on dough's characteristics during processing as well as its suitability for certain end uses. In addition, levels and types of selected ingredients affect the water and gluten interaction differently, i.e. ingredients do not all function in the same manner. Another concern with dough and batter compositions is that they must be formulated to produce different desired characteristics for different products. In other words, a dough used for bread will have different ingredients and levels thereof than doughs made for pretzels or batters made for coating fish filets. As a result, measuring the effects of ingredients on dough and batter characteristics is obviously significant.

Doughs also commonly include ingredients such as salt, shortening, dough strengtheners, sweeteners, enzymes, yeast, oxygenizing agents and reducing agents. These ingredients are combined with flour and water through physical work which ultimately results in a dough (i.e. they are mixed). Depending on the desired end product, the different dough ingredients are commonly present in the following ranges which are given in baker's terms. Water is present from 50-70%, salt from 0.5-3%, sweetener from 1-15%, shortening from 0.5-20%, compressed yeast from 0.5-8%, and vital wheat gluten from 0.5-3%. Common oxidants used in doughs include ascorbic acid which has no set limit but is generally found in an amount of 60-90 ppm, potassium bromate which is generally present in an amount of up to 75 ppm, and ADA which is normally present in an amount up to 45 ppm. As with oxidants, reducing agents such as L-Cysteine are also often used. For example, amounts of L-Cysteine included in doughs range from 20-50 ppm. Finally, emulsifiers, dough strengtheners and crumb softeners such as sodium stearoyl lactylate (SSL), diacetyltartaric acid esters of monoglyceride (DATEM), and mono and diglycerides are also often used. When SSL is used, it is generally present from 0.5-1%. When DATEM is used, it is generally present in the range of 0-1.5%. When mono and diglycerides are used, they are generally present in the amount ranging from 0.5-0.75%. Of course, not all of these ingredients are used in every dough and selection of the appropriate ingredients and levels thereof depends upon the eventual end-use of the dough or batter.

Batters often contain other ingredients such as salt, shortening, sweeteners, egg, milk, and chemical leavening agents. As with doughs, batters are formed by combining selected ingredients with flour and water through physical mixing which ultimately results in a batter. For batters, water is generally present in an amount ranging from 80% to 140%, salt from 1% to 5%, sweetener from 80% to 150%, shortening from 0% to 60%, chemical leaveners, such as baking soda or bicarbonate of soda, from 0% to 8%, egg from 10% to 70%, non-fat dried milk from 0% to 25% and defatted soy flour from 0% to 10%. Once again, selection of the appropriate ingredients and levels thereof is dependent upon the eventual end-use or end product desired.

Ingredient selection is another major factor for determination of dough and batter characteristics as well as the eventual suitability for a desired product. Many kinds of chemical and physical reactions which occur during dough processing depend on the effect each ingredient exerts on water and/or gluten during dough and batter formulation. Thus, dough formulation and processing for desired end product and end product quality is positively related to types and levels of each ingredient. Each ingredient has its own specific functionality for the dough or batter characteristics. Different ingredients or quantities of ingredients may affect various types and grades of flours differently. The effects are often related to properties or characteristics of the flour and protein contained therein as well as the water content of the dough or batter. Additionally, interaction among the various ingredients may also impact dough and batter characteristics during dough and batter development.

During dough and batter processing, the ingredients are subjected to different magnitudes of force and those forces are applied at different rates. At the same time, the dough or batter is subject to physical changes resulting from chemical reactions taking place at the molecular level. In other words, the chemical reactions which occur during processing directly effect the changing product characteristics. In contrast to the physical changes which occur and are perceptable by sight and touch, these chemical reactions are not directly perceptable. Therefore, measurements of characteristics based on chemical change is much more challenging than measurements based on physical change. Moreover, these measurements of chemical changes may display more detail as to how each ingredient effects the dough and batter characteristics.

An overriding concern for manufacturers of doughs and batters is to consistently produce dough or batter-based products which are of optimum quality. Consistent production of optimum quality dough and batter based products is difficult because of the differences lot-to-lot in starting ingredients (e.g., flour) and also variations in ingredients or amounts used. To assist in the consistent production of optimum quality dough and batter products, methods have been developed which measure physical characteristics of doughs and batters as they change during progression through the different production stages.

Dough characteristics and effects of ingredients have been measured by monitoring changes in physical characteristics of dough during formulation (mixing) thereof. These differences in characteristics and the effects of different ingredients have conventionally been measured using resistance-measuring apparatuses such as a Labtron Mixer System (American Ingredients Company, Kansas City, Mo.) which measures temperature and torque as dough is mixed using strain gauges attached to the mixer. Alternatively, a mixer's power consumption can also be measured in order to give information regarding dough characteristics. It is known that as dough resistance increases, power consumption will likewise increase. Alternatively, changes in dough characteristics during dough development in large mixers can be measured using a probe linked with a load cell that measures the force exerted by dough moving around the mixing bowl.

There have also been other methods and instruments available to estimate and measure an ingredient's effect on dough characteristics, especially a given dough's physical properties and the complex rheological behavior thereof. These instruments include the Mixograph, Farinograph, Extensigraph, and Alveograph, all of which have been used to physically measure dough characteristics as part of rheological behavior. Another method is dynamic rheological measurement using the Rheometer. Methods for measuring ingredient functionality on dough and batter characteristics using these types of instruments were developed based on the torque and energy input (i.e. physical characteristics) necessary for mixing the tested doughs and batters. All of the aforementioned instruments are non-invasive and measure dough characteristics in real time, however, these methods do not provide a way to measure the molecular and chemical changes which occur in a dough during its processing. Dough characteristics measurable by the foregoing instruments and methods include dough development, dough mixing tolerance, dough elasticity or dough resistance to flow or extension, dough extensibility, or dough stress or strain, etc., all of which depend upon physical properties and, thereby, the chemical reactions which occur during dough mixing.

Additionally, many physical and chemical reactions which occur during dough and batter processing are related to gluten or water properties which change during mixing due to interactions with each other and with ingredients added during mixing. It is known that water content of doughs and batters is important in the mixing process as well as in the baking process (W. Bushuk, Distribution of Water in Dough and Bread, 40(5) Baker's Digest, p. 38 (1966)) (the teachings of which are incorporated by reference herein). Some of the foregoing instruments and methods were designed to determine the amount of mixing a dough requires to reach a predetermined optimum mixture at any stage during the mixing process or the amount of water that should be added to the flour in order to facilitate producing an optimum dough or batter. They were also found useful in characterizing the various flours or other ingredients (Hosney, R. C., Rheology of Doughs and Batters, Principles of Cereal Science and Technology, Am. Assoc. of Cereal Chemists, St. Paul, Minn., p. 213 (1994)) (the teachings of which are incorporated by reference herein). However, it is important to note that changing dough and batter characteristics are attributable to dough ingredients and formulation only if the mixer performance and environment remain constant.

Methods used to measure chemical changes in doughs during mixing include NIR which has been used to monitor dough characteristics during dough mixing (Wesley, I. J., et al., 27 Non-Invasive Monitoring of Dough Mixing By Near-Infrared Spectroscopy, Journal of Cereal Science, 61-69 (1998)) (the teachings of which are incorporated by reference herein). In general, the origin of NIR is the overtone and combination bands of fundamental vibrations in the mid-infrared spectrum from 2500 to 15000 nm (Wetzel, D. L., Analytical Near Infrared Spectroscopy, Instrumental Methods in Food and Beverage Analysis, Elsevier Science, B. V., Wetzel, L. B. D., and Charalambous, G., eds., p. 141 (1998)) (the teachings of which are incorporated by reference herein). When a compound is in different environments, its characteristic absorption band will shift to other wavelengths because the frequency depends on the force constant and force constant varies with disturbance of the bonding caused by the presence of other competing groups. It is well known that salt has no absorption in the near infrared, yet salt can be detected by its effect on water absorption, whose bands shift from one location to another. Most ingredients, like salt, in dough and batter formulas directly affect water physical and chemical properties such as water's absorption rate into flour.

In the dough system, the ratio of free water to bound water as well as water physical and chemical property changes with dough mixing can be detected by the NIR spectrometer. Those water property changes may be related to the dough and batter characteristics. It is well known that there is no “free” water available in the dough system at the time when dough is completely developed. The second derivative maximum at 1380 nm is highly related to water absorption in the dough system and the wavelengths around the 1380 nm are also highly related to water absorption. In other words, the peak at 1380 nm is located in middle of certain wavelengths that are related to water absorption in the dough system. This peak is a mathematical artifact of the method of computation of the second derivative for the large water peak observed in the raw spectrum. Therefore, the wavelength at 1380 nm can be used to indicate the dough system changes including chemical and physical properties.

Using NIR spectroscopy to measure characteristics of doughs or batters is more complicated than using NIR spectroscopy to measure characteristics of dry materials or materials having a low water content. First of all, the high water content in the dough or batter may prevent other measurements of chemical reactions in the dough. Secondly, the chemical reactions are continuously changing, which means that the types and quantities of certain compounds are not constant. Finally, spectral noise during measurements effects the accuracy of results of the spectral evaluation. Moreover, all spectral data collected by NIR spectroscopy is not easily viewed by the naked eye (differences are not necessarily visible) despite the fact that there are differences between doughs, especially doughs having different ingredients and levels thereof.

Raw spectral data collected by NIR spectroscopy cannot be easily used to view and explain how different ingredients affect dough properties even after the raw spectral data was processed for the second derivatives and timeplot (FIG. 1). If one had diffuse reflectance spectra of high protein wheat flour and lower protein wheat flour plotted as the log1/reflectance vs. wavelength, the difference in the protein levels between the high and low protein samples would be hidden within the width of the pen used to plot each spectrum (Wetzel, D. L., Analytical Near Infrared Spectroscopy, Instrumental Methods in Food and Beverage Analysis, Elsevier Science, B. V., Wetzel, L. B. D., and Charalambous, G., eds., p. 141 (1998)) (the teachings of which are incorporated by reference herein). Therefore, the raw continuous wavelength (400 nm to 1700 nm) spectra may not directly be used to specify the dough mixing at a given time during dough mixing. Other data processing techniques at specific wavelengths must be developed to view and demonstrate the effects of ingredients on dough characteristics.

Therefore, what is needed is a method of processing data collected by the NIR spectroscopy which distinguishes and magnifies differences between doughs and batter based products and permits easy observation of these differences. What is further needed is a non-invasive real time method for the determination of the effects of different ingredients, and levels thereof, on physical and chemical characteristics of doughs and batters for bakery products during mixing using NIR spectroscopy. Finally, what is needed is a method of measuring chemical changes in a dough or batter, monitoring and measuring the effects of different ingredients and levels thereof which allows an optimum flour, dough or batter product to be consistently prepared despite differences in initial flour quality, ingredients, levels of ingredients, or mixing times which previously resulted in doughs and batters of dramatically different quality.

### SUMMARY OF THE INVENTION

The present invention provides methods for evaluating dough or batter characteristics during formulation thereof, for identifying flour differences, and for determining ingredient functionality. Specifically, the effects of ingredients and levels thereof on the physical and chemical characteristics on doughs and batters for bakery products during mixing are preferably determined using a diode array NIR spectrometer with certain spectral data processing, transformation, and arithmetic computations. The processed and transformed spectral data is plotted against dough mixing time. The plot is defined as a development plot, which can be used to specify and explain dough characteristics based on the line configuration. Some specific wavelengths or area of specific wavelengths are highly correlated to specific ingredients and may be used to establish algorithm models for a computerized NIR spectrometer installed in bakery product lines to monitor and control the dough characteristics during mixing.

As used herein, the following definitions will apply: “dough” refers to a complex, homogenous, and viscoelastic mass of diverse ingredients including, at the least, flour and water and potentially having the same ingredients and levels thereof described in reference to the prior art; “batter” is a complex, homogenous, flowable and viscous mass of ingredients which includes, at the least, flour and water and potentially having the same ingredients and levels thereof as described in reference to the prior art. Unless noted otherwise, when doughs are mentioned, it is understood that batters are also contemplated.

The present invention provides accurate, efficient, real-time and non-invasive methods for the determination of the effects of ingredients and quantities thereof on physical and chemical characteristics of doughs and batters for bakery products during mixing using NIR spectroscopy. The doughs includ mixed dough, sheeted dough, and any other dough composed of any grain flour and water. Log1/R measurements at 1380 nm were chosen as a measure of the effect of ingredients on dough and batter properties, although other wavelengths may be used if desired.

The preferred NIR spectroscopy instrument is a DA-7000 NIR/VIS spectrometer made by Perten Instruments North America, Inc., which is a continuous spectrum post-dispersive, diode array based, dual-channel, computerized near-infrared/visible spectrometer. The present invention advantagously uses data collected at the 1380 nm wavelength, and then processes this data using certain spectral data processing, arithmetic computations, and may generate development plots for ease of understanding. The raw spectra (log1/R) is then preferably converted from absorbance units to Kubelka-Munk units. The Kubelka-Munk equation is perhaps the best known relationship for diffuse reflectance (Wetzel, D. L., Analytical Near-Infrared Spectroscopy, Kansas State University, Manhattan, Kans., course notes, pp. 2-1 to 2-10 (1989)) (the teachings of which are hereby incorporated by reference). Next, the spectra as Kubelka-Munk units are processed to determine the second derivatives thereof using the Savitsky-Golay method, based on a second degree polynomial and 11 point smoothing. The second derivative spectra are then expressed as a plot of mixing time versus spectral data, which is herein referred to as a “time plot” at 1380 nm. The time plot curve is further smoothed using a second degree polynomial with 11 point smoothing. The smooth time plot curve XY is then used to calculate the cumulative transformed Kubelka-Munk units, which are used in a plot; that is, in a development plot between cumulative Kubelka-Munk units and mixing time. This development plot is then used to view changes in dough and batter characteristics and how the ingredients and levels thereof effected dough and batter properties during mixing.

Use of NIR spectroscopy coupled with the preferred data processing has been found to illustrate and define the effects of different water absorption levels on otherwise identical dough compositions. Therefore, optimum water absorption levels for different dough applications can be determined using NIR. The methods of the present invention are also useful with respect to ingredients, levels thereof, and their effects on dough development and ultimate product quality. Differences between dough compositions differing only in the variety of flour used can also be discerned. Knowledge of these differences permits preadjustment or inclusion of ingredients and levels thereof which will interact optimally with the flour variety in order to produce an optimum dough product. In other words, different varieties of flours may require different levels of water absorption in order to produce products having development plots exhibiting certain desired characteristics at certain stages of dough development. Based on such ascertained flour and dough characteristics, the dough production process can also be adjusted. For example, some flours require longer hydration times or development times.

The effects of using ingredients such as vital wheat gluten, salt, reducing agents, sweeteners, shortening, and oxidizing agents may also have an impact on dough development, and these effects can be determined using methods of the present invention.

Specifically, the present invention provides methods of analyzing wheat-based dough or batter products by mixing dough or batter ingredients together, wherein the dough or batter products include flour and water and at least one additional conventional dough or batter ingredient, performing near infrared analyses of the products at different times during mixing and comparing the analysis in order to indicate the effect of the amount or type of ingredient added. The near infrared analyses can be carried out at the same wavelength with a preferred wavelength being 1380 nm. Data derived at specific near infrared wavelengths from absorbance measurements is used to generate a mixing time plot for each of the products. This derived data is processed by converting the spectral data to Kubelka-Munk units, determining the second derivative of the KM units, graphing the second derivatives versus mixing time, smoothing the resultant curve, and cumulating the sum of the respective transformed KM units in order to generate a development plot between these cumulative transformed KM units and mixing time. The resultant analysis can then be used to identify differences between doughs having different ingredients and levels thereof and the impacts these differences had on dough development. The present invention also identified wavelengths which were correlated to certain ingredients of the doughs and batters tested. This type of identification allows one to monitor specific wavelengths and ascertain dough ingredient inclusion, level of ingredient, or effect of the ingredient or level thereof. Finally, the present invention provides an apparatus for analyzing cereal grain-based dough or batter products. This apparatus includes a mixing container, preferably a bowl having a window in the bottom or sidewall through which radiation can be emitted and detected, and a near infrared spectrometer having a radiation emitter and a radiation detector.

### BRIEF DESCRIPTION OF THE DRAWINGS

FIG. 1 is a graph illustrating second derivative log (1/R) versus mixing time near infrared (NIR) timeplots for three full formula doughs having different water absorption levels, where the raw spectral data was used without Kubelka-Munk (KM) conversion, as described in Example 1;

FIG. 2 is a graph illustrating second derivative KM units versus mixing time for the three doughs of FIG. 1, where the raw NIR spectral data was converted to KM units, as described in Example 2;

FIG. 3 is a graph illustrating cumulative second derivative KM units versus mixing time for the three doughs of FIG. 1, where the raw NIR spectral data was first converted to KM units and then second derivative computed and then smoothed and cumulated, as described in Example 3;

FIG. 4 is a graph illustrating two development plots for respective doughs, where the plots were generated using the preferred conversion of raw NIR spectral data to KM units followed by smoothing and cumulation thereof, with the phases of dough development marked for each plot, as described in Example 4;

FIG. 5 is a graph of Labtron units versus mixing time for three test doughs containing 0%, 1% and 3% of vital wheat gluten, as described in Example 5;

FIG. 6 is an illustration of the preferred Labtron mixing system, modified to accommodate an NIR probe in the mixing bowl;

FIG. 7 is a graph illustrating two dough sample mixing time plots generated using the preferred NIR procedure of the invention, with the doughs varying in water absorption value, as described in Example 7;

FIG. 8 is a graph depicting the effect of salt content and water absorption on various doughs, using the preferred NIR analysis of the invention, as described in Example 8;

FIG. 9 is a graph illustrating two test dough mixing time plots and demonstrating the effect of L-cysteine on dough characteristics, using the preferred NIR analysis of the invention, as described in Example 9;

FIG. 10 is a graph illustrating test dough mixing time plots for the doughs of FIG. 9, wherein the doughs were analyzed using conventional Labtron analysis and graphed using cumulative Labtron units, as described in Example 9;

FIG. 11 is a graph illustrating the test dough mixing time plots for the doughs of FIG. 5, wherein the doughs were analyzed using the preferred NIR analysis of the invention, as described in Example 5;

FIG. 12 is a graph illustrating differences and similarities in dough mixing time plots for doughs having identical compositions and varying only in the variety of flour used, wherein two of the three doughs used identical flours and wherein the doughs were analyzed using the preferred NIR analysis of the invention, as described in Example 10.

FIG. 13 is a graph illustrating correlation coefficients relating water absorption and area under wavelengths for two commercial flours, as determined by the preferred NIR analysis of the present invention, and as described in Example 16;

FIG. 14 is a graph illustrating dough mixing time plots for three full formula doughs varying only in the amount of sugar added to each dough, wherein the doughs were analyzed using preferred NIR analysis of the invention, as described in Example 11;

FIG. 15 is a graph illustrating dough mixing time plots for three full formula doughs varying only in the amount of shortening added to each dough, wherein the doughs were analyzed using preferred NIR analysis of the invention, as described in Example 12

FIG. 16 is a graph illustrating dough mixing time plots for two full formula doughs varying only in the amount of potassium bromate added to each dough, wherein the doughs were analyzed using preferred NIR analysis of the invention, as described in Example 13

FIG. 17 is a graph illustrating dough mixing time plots for four full formula doughs varying only in the amount of ADA added to each dough, wherein the doughs were analyzed using preferred NIR analysis of the invention, as described in Example 14;

FIG. 18 is a graph illustrating the effect of water absorption on dough properties in full formula doughs having different varieties of commercial strong bread flour dough, wherein the doughs were analyzed using the preferred NIR analysis of the invention, as described in Example 15;

FIG. 19 is a graph illustrating a conventional Labtron curve graphing Labtron units versus mixing time for two different varieties of flour, as described in Example 4;

FIG. 20 is a graph illustrating a conventional Labtron curve graphing Labtron units versus mixing time for three doughs having identical compositions and varying only in the variety of flour used, as described in Example 10; and.

FIG. 21 is a graph illustrating differences and similarities in dough mixing time plots for doughs having identical compositions and varying only in the variety of flour used, wherein two of the three doughs used identical flours and wherein the doughs were analyzed using the preferred NIR analysis of the invention, as described in Example 10.

### DETAILED DESCRIPTION OF THE PREFERRED EMBODIMENT

The following examples set forth the preferred embodiments of the present invention. It is to be understood, however, that these examples are provided by way of illustration and nothing therein should be taken as a limitation upon the overall scope of the invention.

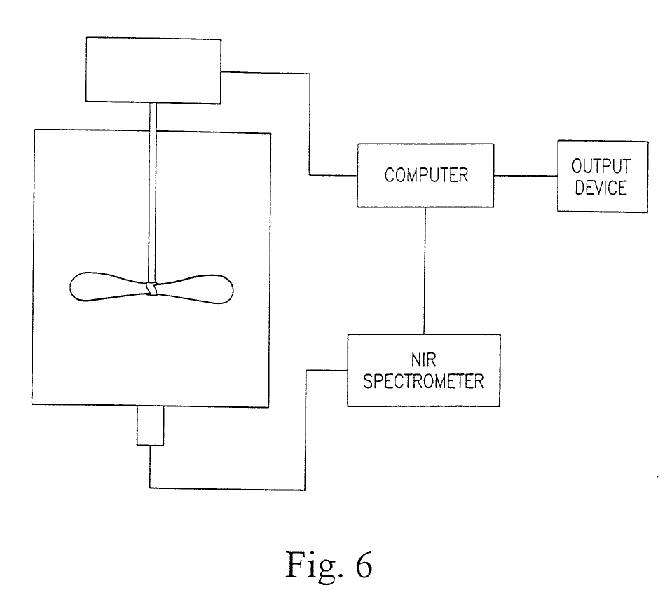

Unless noted otherwise, the following mixing system and set-up protocol was used for all examples. A Labtron Mixer System (American Ingredients, Inc., Kansas City, Mo.) was used. This system consisted of a Labtron apparatus (Labtron) which includes a Hobart mixer (Model A-200T, Hobart Corporation, Troy, Ohio) having a double helical agitator and a jacketed bowl connected to a chilled water system set at 78° F. This apparatus is illustrated in FIG. 6. The Labtron apparatus is a completely self-contained dough mixing unit which has been designed for use in laboratory settings. The Labtron continuously monitors changes in torque and temperature of a dough as it is mixed by its uniquely designed mixing arms. Results are graphed on a computer screen using the data generated by the Labtron's monitoring of torque required to mix the dough and temperature. This graphed data can be compared with data obtained from previously mixed doughs thereby showing any differences in the mixing curves which might have resulted from changes in flour strength, absorption requirements and other ingredients.

A diode array near-infrared (NIR) spectrometer, DA-7000 (Perten Instruments, Inc., Springfield, Ill.), capable of detecting diffusely reflected radiation from 400-1700 nm, was used in these examples. The NIR spectrometer was connected to the mixer via a fiber optic probe positioned beneath a window on the bottom of the mixing bowl. The DA-7000 NIR spectrometer is a continuous spectrum, post-dispersive, diode array based, dual-channel, near-infrared/visible spectrometer. Fiber optics are used to convey the chopped, high-intensity, broad-band energy from the DA-7000's source module to the sample through the illumination fiber. After passing through or interacting with the sample the modified light energy is returned to the detector module through the sensing fiber. The modified energy entering the detector module is dispersed by a stationary diffraction grating, and energy at specific wavelengths is focused on a diode array, which converts the signals of energy into a digital format. The digital signals are then fed to a system board within the DA-7000 system cabinet where they are processed for transmission to the digital signal processing board within the DA-7000's computer. Subsequently, this data can be analyzed by a computer having software packages designed to simplify the data collection, processing, analysis and storage.

The set-up protocol included warming up both the NIR spectrometer and the Labtron for more than one hour before any dough was mixed, as recommended by the operation manuals for both machines. Next, noise and baseline tests were performed in order to verify that the tests to be performed would be valid tests. These tests were performed before the NIR spectrometer was used to measure dough characteristics.

To perform sample testing, the mixing bowl was cleaned out, a project name was selected for the data collection and the baseline number was recorded to identify each sample. All of the dough ingredients except for water were added to the mixing bowl and the settings of the NIR spectrometer and Labtron were rechecked to ensure proper operation. This entailed rechecking and verifying that everything was hooked up properly and ready to go before adding water to the mixer bowl and starting the mixer.

Next, the appropriate amount of water was added to the dry ingredients in the mixing bowl, and dough mixing was begun. During mixing of each dough sample, the NIR spectrometer collected spectral data which was subsequently manipulated to obtain time plots.

Both the Labtron data and the NIR spectral data were collected in the same manner. The NIR spectral data was collected at approximately one second intervals. NIR data at the 1380 nm wavelength was chosen for analysis purposes because this wavelength may be related to water absorption in a dough system. However, any wavelength between 400 nm and 1700 nm could be used to practice the present invention. This raw data were analyzed and converted by the Grams32 version 5 computer program by Galactic Industries Corp. (Salem, N.H.) using the following conversions. First, the raw spectra were converted from absorbance units to Kubelka-Munk (KM) units. Next, the KM spectral were processed for the second derivative KM spectra based on the Savitsky-Golay method using the second degree polynomial and 11 point smoothing. These second derivative datapoints were expressed as a plot of time mixed (X) vs. spectral data (Y), herein referred to as a “time plot” at 1380 nm. This time plot curve was further smoothed using a second degree polynomial with 11 point smoothing (based on the Savitsky-Golay method). The data from the time plot curve (XY) was used to calculate the cumulative second derivative KM units, i.e. the sum of the respective transformed KM units, which could be used in a development plot between cumulative transformed KM units and mixing time. The resulting development plot is then reviewed and used in assisting in interpreting characteristics of the dough and how different ingredients affected certain dough properties during mixing.

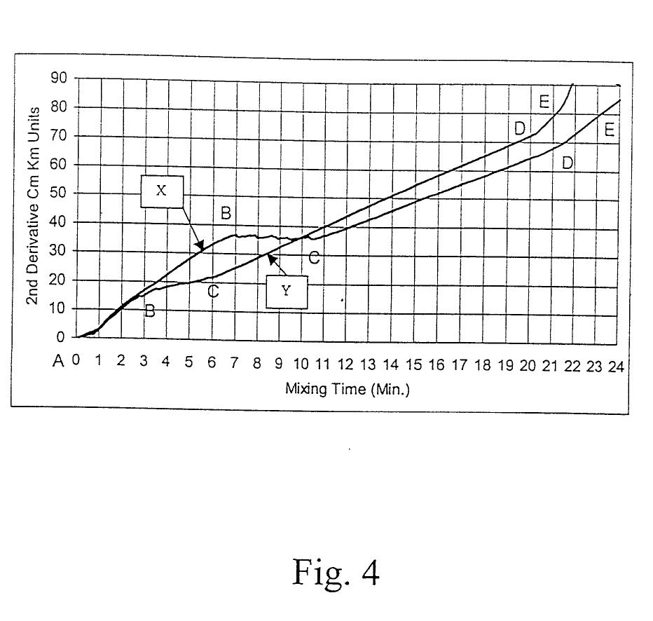

There are four phases in a normal dough development plot line (e.g., see FIG. 4). The AB phase represents the dough hydration period. The BC phase represents the dough development period. The CD phase represents the dough over-development period. Finally, the DE phase represents the phase in which the dough starts to stick to the bottom of the mixing bowl. These different phases, represented in the development plot, can be used to explain dough characteristics during mixing. For example, the hydration time for doughs made with high levels of water is comparatively longer than that for doughs with low levels of water. Conversely, the development time for doughs with high levels of water is shorter when compared with doughs with low levels of water. The total mixing time for doughs with high levels of water is longer when compared with doughs with low levels of water.

Additionally, unless noted otherwise, percentages of ingredients added to the doughs are provided in conventional “bakers' percent” terms. Bakers' percent is defined herein as the weight of individual ingredients expressed as a percentage of the weight of flour in the formula. For example, if a sample had 100 g flour, 15 g salt, 15 g sugar, 5 g shortening and 2 g compressed yeast, the sample would contain 15% salt, 15% sugar, 5% shortening and 2% compressed yeast when calculated using conventional bakers' percent terms.

### EXAMPLE 1

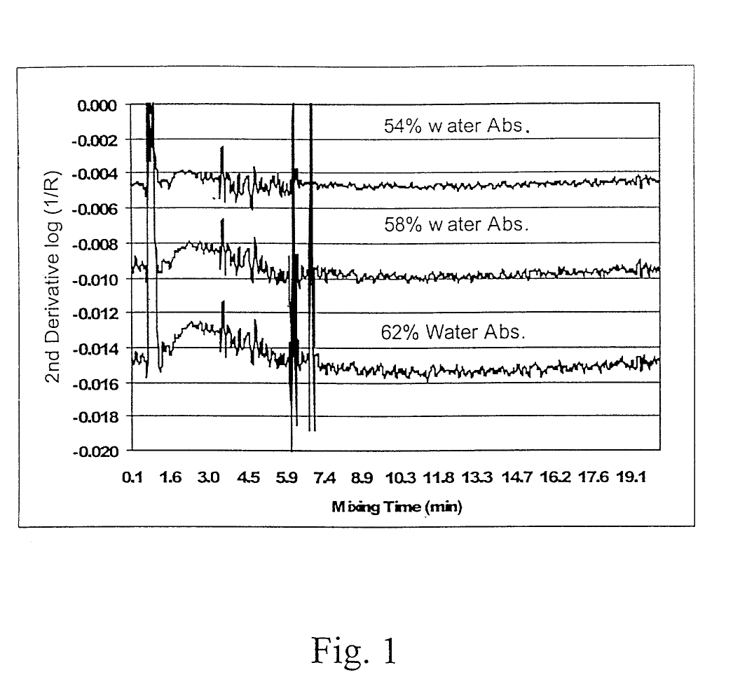

In this comparative example, a series of three doughs were prepared and analyzed as set forth above, except that the raw NIR spectral data were not transformed to KM units, but rather were plotted directly as second derivative log(1/R) versus mixing time. The resultant time plots demonstrated the practical advantages of the preferred KM transformation, insofar as the analysis of the plots is concerned. Specifically, three full formula doughs were formed, each comprising 1100 g Hard Red Winter Wheat flour, 77 g sugar, 33 g shortening, 22 g compressed yeast, 22 g salt and either 54%, 58% or 62% water absorption. Raw spectra were processed by the following steps: a) raw spectra were selected for each sample; b) the second derivative of these spectra were computed using the Savitsky-Golay method at second degree polynomial and 11 point smoothing; c) a time plot between absorbance of 1380 nm wavelengths was constructed; and d) the time plot curve was further smoothed using the Savitsky-Golay method at second degree polynomial and 11 point smoothing. Respective time plots (smoothed using the Savitsky-Golay method) were then constructed and graphed using this calculated derivative data, and are shown in FIG. 1.

As can be observed from the consideration of the FIG. 1 time plots, the plots do not clearly depict the various phases of dough development, nor do they clearly depict differences among the doughs. Therefore, these time plots are thus essentially useless as analysis tools.

### EXAMPLE 2

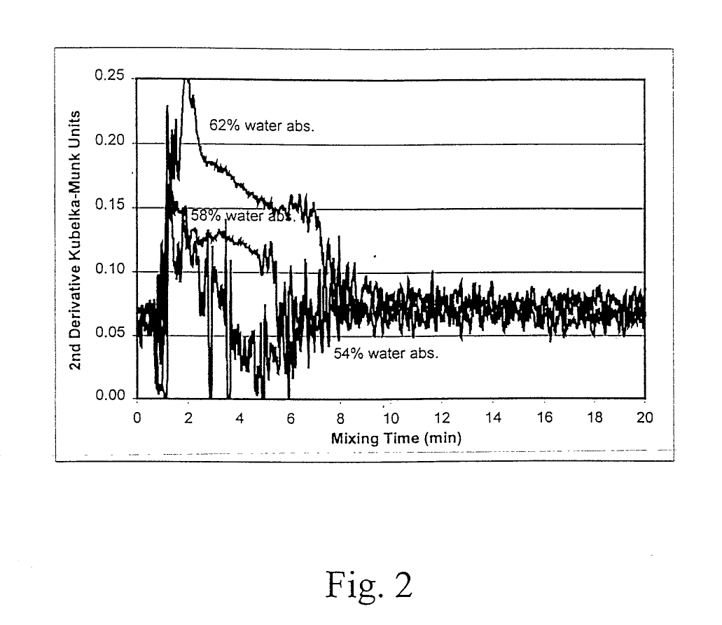

This example demonstrates that converting the raw spectral absorbance units to KM units before further data processing produces graphs which are more discriminating than those shown in FIG. 1. In particular, the raw spectral data from Example 1 was first converted to KM units and these were then processed as described above to produce smooth second derivative time plots. These plots are depicted in FIG. 2, where it will be seen that the various phases of dough development can be more readily discerned, as compared with the FIG. 1 plots.

### EXAMPLE 3

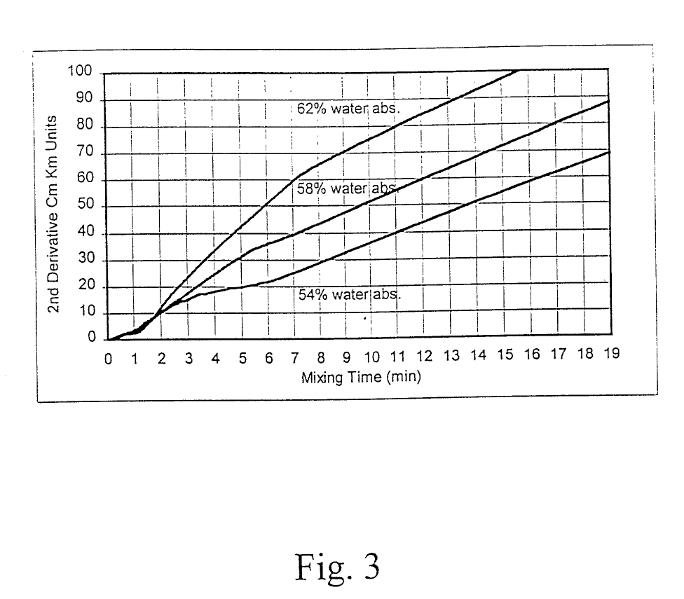

In this example, the second derivative KM unit data from Example 2 was cumulated as described and graphed as cumulative second derivative KM units versus mixing time. These time plots are shown in FIG. 3, where it will be seen that the phases of dough development are very readily discernible and that the three plots are separated for easy analysis and comparisons among the doughs. For example, the FIG. 3 plots show that the 54% water absorption dough had a shorter hydration period (AB as shown in FIG. 4) as compared with the 62% water absorption dough; moreover, the 62% water absorption dough had a longer dough mixing time for development (from A to C as shown in FIG. 4).

### EXAMPLE 4

In this example, full formula doughs made using two different varieties of Hard Red Winter Wheat were prepared and tested. The dough formulas were identical except for in one dough X, Karl 92 flour was used, whereas in the other dough Y, variety 2137 flour was employed. Each dough had: 1100 g Hard Red Winter Wheat, 77 g sugar, 33 g shortening, 22 g compressed yeast, 22 g salt at a 54% level of water absorption. As shown in FIG. 4, the doughs, differing only in the variety of Hard Red Winter Wheat used, exhibited significant differences in their development plots. Dough X had relatively longer dough hydration (AB) and dough development (BC) periods while dough Y had a relatively shorter dough hydration period (AB) and dough development period (BC).

The configuration of the development plot, such as the one shown in FIG. 4, may be affected by the amount of “free” water in the dough during mixing. When mixing a dough in the AB phase, the amount of “free” water decreases up to point B at which there is no “free” water. However, just because there is no “free” water at this point does not mean the dough cannot be further developed. In fact, the dough maybe further developed when the “bound” water is redistributed from non-uniform to uniform distribution in the dough system. This redistribution accounts for the nearly constant slope of the configured line during the BC phase. At point C, when the dough with a certain water level is developed, the distribution of water in the dough system should be nearly uniform. After point C, when the dough is overmixed, some of the “bound” water is released (i.e. when the gluten alignment changes thereby increasing “free” water). The availability of this “free” water accounts for the steady increase of the configured line during the CD phase of dough development. Once the mixing time passes the D point, dough begins to coat the bottom of the mixing bowl thereby making more “free” water available on the dough surface. Once again, this increase in “free” water is due to the realignment of gluten in the dough and/or other chemical bonds replacing “bound” water. This release of “free” water accounts for the increase of the configured line during the DE phase which indicates dough break down.

### EXAMPLE 5

In this example, three doughs were prepared and Labtron curves and time plots in accordance with the invention were generated for each dough. Each dough contained 1100 g Hard Red Winter Wheat flour, 77 g sugar, 33 g shortening, 22 g compressed yeast, 22 g salt and 62% water. Two of the doughs were supplemented with vital wheat gluten (1% and 3%, respectively), whereas the final dough contained no vital wheat gluten supplement.

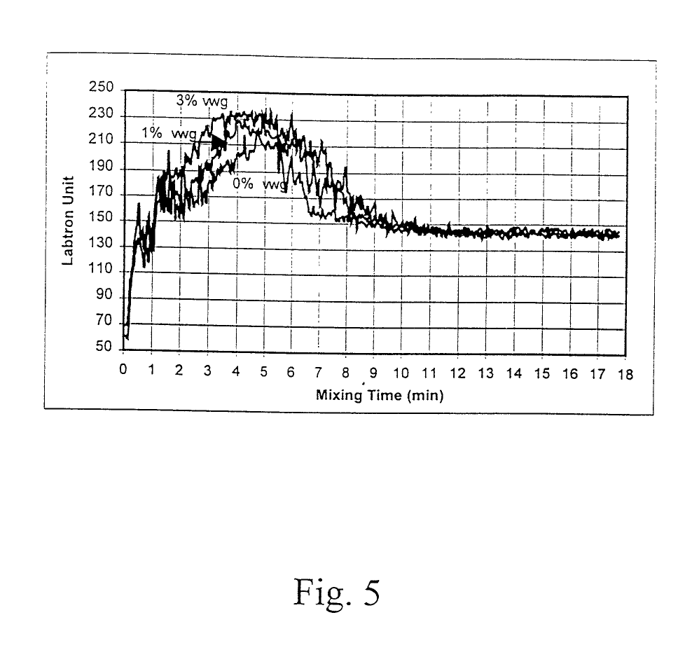

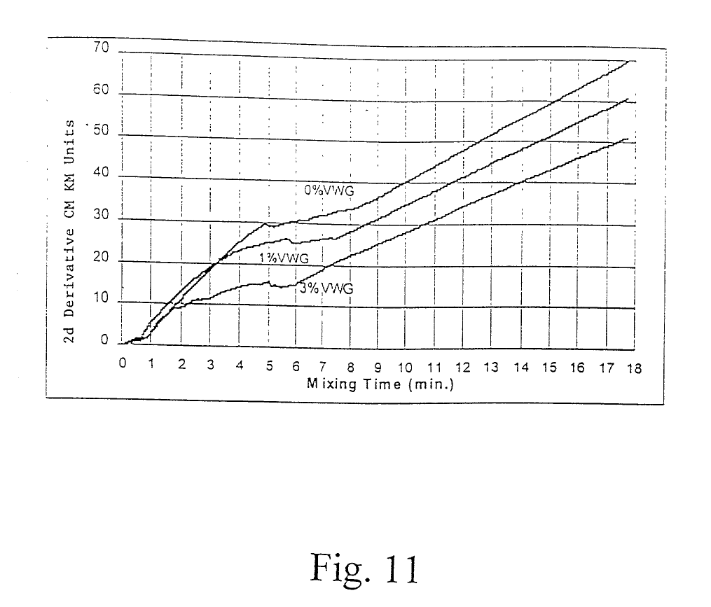

FIG. 5 illustrates the Labtron curves for these doughs. It is apparent that there are differences between the three doughs, however, it is difficult to determine at which phase(s) of dough development the differences occur. Comparative FIG. 11 sets forth the cumulative second derivative KM time plots for these three doughs. It is readily apparent that the FIG. 11 plots are much easier to read and analyze than the Labtron curves.

When comparing the Labtron results (FIG. 5) to the NIR results (FIG. 11), differences among the doughs having three different levels of vital wheat gluten are much easier to discern in FIG. 11 than in FIG. 5. Furthermore, the demarcations between the different dough development phases are easier to discern in FIG. 11 than in FIG. 5. For example, one can discern that the dough hydration period (corresponding to the AB line from FIG. 4) takes a little less than five minutes for the dough with no added vital wheat gluten, about four minutes for the dough with 1% added vital wheat gluten and slightly less than two minutes for the dough having 3% added vital wheat gluten. Similarly, the dough development period (corresponding to the BC line from FIG. 4) is also much easier to discern in FIG. 11 than in FIG. 5.

Table 1 sets forth the NIR hydration and development time results:

TABLE 1
Effect of Vital Wheat Gluten (0, 1, and 3% levels)
on Dough Characteristics with Two Levels of Water Absorption

| Water | | | | |
|---|---|---|---|---|
| Absorption | Vital Wheat | Hydration | Development | Total Mixing |
| (%) | Gluten (%) | Time (min) | Time (min) | Time (min) |
| 58 | 0 | 4.7 | 2.8 | 7.5 |
| 58 | 1 | 2.3 | 4 | 6.5 |
| 58 | 3 | 1.7 | 4.3 | 6 |
| 62 | 0 | 4.9 | 3.3 | 8.2 |
| 62 | 1 | 4 | 3.5 | 7.5 |
| 62 | 3 | 1.8 | 4.3 | 6.1 |

### EXAMPLE 6

This example correlates dough mixing times as determined by Labtron and NIR for 37 dough samples. Results from this testing are given in Table 2. The correlation coefficient after 37 full formula dough samples was 0.944. Each dough sample tested differed from the other doughs in their formulations.

Unless noted otherwise, these doughs contained 1100 g Flour, 22 g compressed yeast, 22 g salt, 33 g shortening, 77 g sugar (sucrose), and a variable amount of water. Differences in dough ingredients are noted in Table 2 which also provides optimum dough mixing times as determined by a Labtron and an NIR spectroscopy apparatus.

TABLE 2

| | Labtron | NIR | |
|---|---|---|---|
| | Mixing | Mixing | |
| Point in | Time | Time | Flour Type, Ingredient Differences, |
| FIGURE | (min.) | (min) | Water Absorption Level |
| 1 | 3 | 3.6 | 7853 Flour, 58% Water Absorption |
| 2 | 3.5 | 3.8 | Commercial Flour, 40 ppm L-Cysteine, |
| | | | 62% Water Absorption |
| 3 | 3.7 | 3.5 | 7853 Flour, 0% Salt, 62% Water |
| | | | Absorption |
| 4 | 4.8 | 4.9 | Commercial Flour, 0% L-Cysteine, 62% |
| | | | Water Absorption |
| 5 | 5.4 | 7.3 | Tomahawk Flour, 7% Sugar, 62% Water |
| | | | Absorption |
| 6 | 5.5 | 8.5 | Tomahawk Flour, 0% Sugar, 58% Water |
| | | | Absorption |
| 7 | 5.7 | 6.7 | Tomahawk Flour, 0% Sugar, 62% Water |
| | | | Absorption |
| 8 | 6 | 6.5 | 2163 Flour, 60% Water Absorption |
| 9 | 6 | 6 | 2137 Flour, 54% Water Absorption |
| 10 | 6 | 7.5 | 7853 Flour, 2% Salt, 58% Water |
| | | | Absorption |
| 11 | 6 | 8 | 7853 Flour, 2% Salt, 58% Water |
| | | | Absorption |
| 12 | 6 | 8.5 | Tomahawk Flour, 0% Sugar, 58% Water |
| | | | Absorption |
| 13 | 6 | 6.8 | Tomahawk Flour, 7% Sugar, 58% Water |
| | | | Absorption |
| 14 | 6.5 | 6.6 | 2163 Flour, 64% Water Absorption |
| 15 | 6.5 | 7 | Tam 107 Flour, 64% Water Absorption |
| 16 | 7 | 7.3 | 2163 Flour, 56% Water Absorption |
| 17 | 7 | 6.35 | Tam 107 Flour, 60% Water Absorption |
| 18 | 7 | 6 | 2137 Flour, 58% Water Absorption |
| 19 | 7 | 7.5 | Tomahawk Flour, 14% Sugar, 58% Water |
| | | | Absorption |
| 20 | 8 | 8 | Tam 107 Flour, 68% Water Absorption |
| 21 | 8 | 7.2 | 2137 Flour, 62% Water Absorption |
| 22 | 8 | 8.2 | Tomahawk Flour, 14% Sugar, 62% Water |
| | | | Absorption |
| 23 | 8.3 | 9 | 7853 Flour, 2% Salt, 62% Water |
| | | | Absorption |
| 24 | 9 | 8.9 | Karl Flour, 60% Water Absorption |
| 25 | 9 | 10 | Karl 1 m Flour, 0% Shortening, 58% |
| | | | Water Absorption |
| 26 | 9 | 10.2 | Karl 1 m Flour, 0% Shortening, 58% |
| | | | Water Absorption |
| 27 | 9.2 | 10.5 | Karl 1 m Flour, 3% Shortening, 62% |
| | | | Water Absorption |
| 28 | 9.5 | 10.5 | Karl 1 m Flour, 0% Shortening, 62% |
| | | | Water Absorption |
| 29 | 10 | 9.8 | Karl Flour, 64% Water Absorption |
| 30 | 10 | 9.8 | Karl 92 Flour, 50% Water Absorption |
| 31 | 10 | 10.3 | Karl 1 m Flour, 3% Shortening, 58% |
| | | | Water Absorption |
| 32 | 10 | 10.3 | Karl 1 m Flour, 6% Shortening, 58% |
| | | | Water Absorption |
| 33 | 10 | 10.8 | Karl 1 m Flour, 6% Shortening, 62% |
| | | | Water Absorption |
| 34 | 11 | 10.6 | Karl 92 Flour, 54% Water Absorption |
| 35 | 13 | 12.8 | Karl 92 Flour, 58% Water Absorption |
| 36 | 13 | 15 | 7853 Flour, 4% Salt, 58% Water |
| | | | Absorption |
| 37 | 14.4 | 16 | 7853 Flour, 4% Salt, 62% Water |
| | | | Absorption |

Each mixing time in Table 2 represents dough mixing up to point C in the development phase, at which the dough is considered “fully developed.” This high degree of correlation verifies that NIR may be used as a chemical measurement of dough development in the same way that Labtron is used as a physical measurement of dough characteristics. It is also important to note that the determination of dough properties is very important when mixing doughs. When the dough is developed, there is no “free” water available in the dough system for flour particles and other ingredients because all of the water in the system is being used to hydrate the dough system.

### EXAMPLE 7

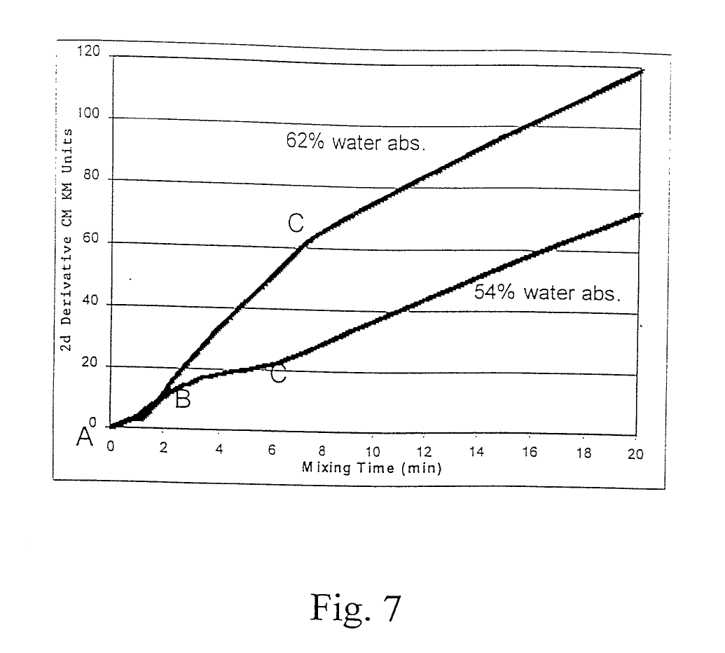

This example demonstrates that optimum water absorption for a dough may be predicted using a development plot based upon NIR spectroscopy. Optimum water absorption is the amount of water used to hydrate the flour particles and other ingredients and which produces the highest quality dough. To predict optimum water absorption, water levels are adjusted until the hydration period (the AB phase of FIG. 4) merges with the dough development phase (the BC phase of FIG. 4) into a continuous AC phase with no discernible line of demarcation between the end of AB phase and the beginning of the BC phase. When these two phases merge, the optimum water absorption level has been exceeded. Therefore, optimum water absorption may be predicted by observing the portion of a time plot just prior to the point where the AB phase merges with the BC phase. The actual optimum water absorption level should result in a very short but discernible development phase (BC phase) in the development plot. Once an optimum water absorption level for a dough has been predicted, this predicted level may be used as the optimum water absorption level for dough mixing. This concept is illustrated in FIG. 7.

In particular, two full formula doughs, the first having 54% water and the second having 62% water and both formed with 1100 g Hard Red Winter Wheat flour, 77 g sugar, 33 g shortening, 22 g compressed yeast, 22 g salt were formed. The lines of demarcation between the difference phases of dough development are much easier to discern in the development curve for the dough having 54% water than in the dough having 62% water. This is because the AB phase has merged with the BC phase into a continuous AC phase. The dough having 62% water no longer has a discernible AB phase indicating that the water absorption has exceeded the optimum water absorption for the dough formula.

### EXAMPLE 8

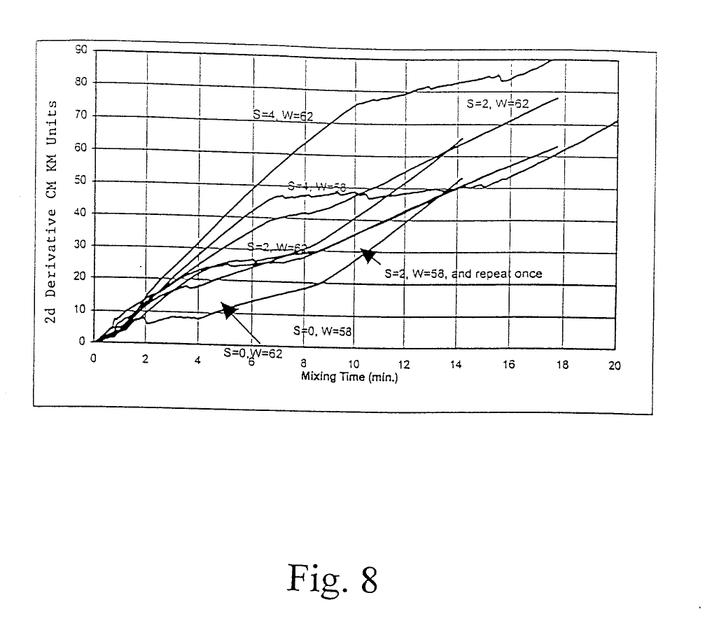

This example demonstrates the effect of salt on dough characteristics wherein doughs having two different levels of water absorption and three different concentrations of salt were tested, and the spectral results were processed in accordance with the preferred procedures of the invention. Results of this experiment are shown in FIG. 8. Six doughs were formed each with 1100 g Hard Red Winter Wheat flour from variety 7853, 77 g sugar, 33 g shortening, 22 g compressed yeast and differing levels of water and salt. The levels of water absorption tested were 58% and 62%. The concentrations of salt tested were 0%, 2% and 4%. As shown by FIG. 8, the development plots for doughs having the same salt concentration but different water absorption levels were almost parallel. In contrast, the development plots for doughs with different salt concentrations at the same water absorption levels intersected, thereby demonstrating that increasing concentrations of salt strongly effected the dough characteristics. These effects were seen during both dough hydration and dough development as both were increased when increasing salt concentrations were used. These results are given in Table 3. Similarly, dough resistances before the doughs were developed were lower for doughs formed with high salt concentrations than for that of doughs formed with low salt concentrations. This can be explained by the presence of salt rendering the gluten relatively more hydrophobic, so that the gluten hydration may be reduced. Thus, doughs formed with higher salt concentrations needed longer hydration times, longer dough development times and exhibited steeper development plots, as compared with doughs formed with lower salt concentrations.

TABLE 3
Effect of Salt on Dough Characteristics

| Water | | | |
|---|---|---|---|
| Absorption | Salt Levels | Dough Hydration | Dough Develop. |
| (%) | (%) | Time (min.) | Time (min.) |
| 58 | 0 | 2 | 4 |
| 58 | 2 | 4.5 | 8 |
| 58 | 4 | 6.5 | 15.5 |
| 62 | 0 | 3.1 | 3.3 |
| 62 | 2 | 6.1 | 8.2 |
| 62 | 4 | 10 | 15.5 |

### EXAMPLE 9

This example demonstrates the effects of L-Cysteine on dough characteristics. L-Cysteine is a reducing compound conventionally known for decreasing dough mixing time. It is believed that L-Cysteine breaks disulphide bonds between gluten proteins by thiol-disulphide interchange thereby causing dispersion of disulphide-bonded aggregates. Under mechanical development conditions, the addition of L-Cysteine softens dough and produces a development curve with a lower relaxation time as compared with untreated flour doughs.

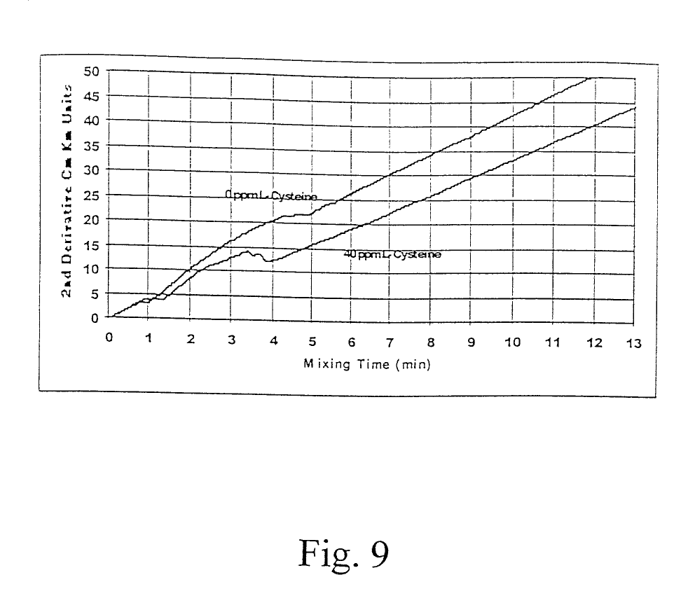

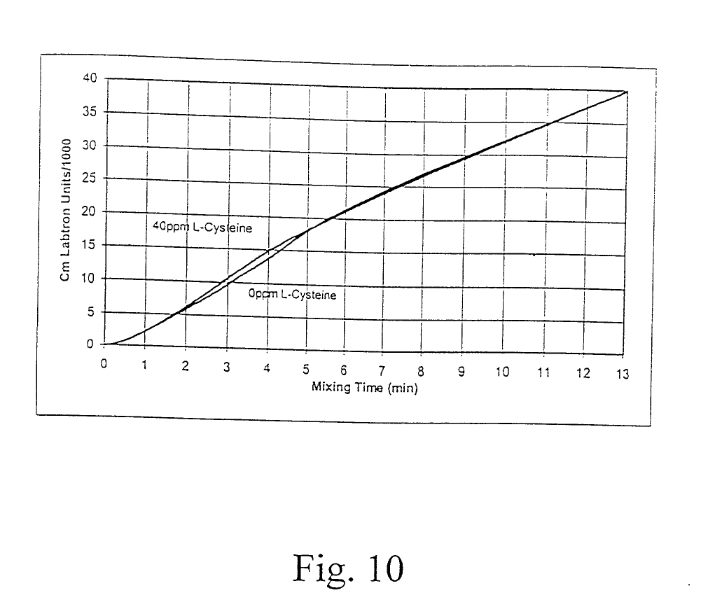

A commercial bread flour was used to test the effect of two different levels of L-Cysteine on dough properties as determined by NIR and the preferred data manipulation steps of the invention, as well as by conventional Labtron analysis. The doughs each included 1100 g of the commercial bread flour, 77 g sugar, 33 g shortening, 22 g compressed yeast, 22 g salt and 62% water absorption. One sample contained no L-Cysteine while the other sample contained 40 ppm L-Cysteine. Addition of the L-Cysteine reduced development time as shown by NIR testing and FIG. 9. FIG. 10 shows the effect of L-Cysteine on dough resistance as determined by the Labtron based on cumulative Labtron units. The resistance of dough with L-Cysteine was higher than the dough without L-Cysteine before the full dough development around five minutes. After the dough was developed, the resistance of dough with L-Cysteine was similar to that of dough without L-Cysteine. NIR can be used to interpret how this additive affects dough properties.

Doughs with L-Cysteine have less “free” water than doughs without L-Cysteine because the gluten in the doughs having L-Cysteine is easily exposed to the water. Therefore, the doughs with L-Cysteine had shorter hydration times. Comparing the work inputs, as shown by the Labtron, there was no difference in the work input after the dough is developed. However, there is more work input for doughs with L-Cysteine than for doughs without L-Cysteine before the dough is fully developed.

### EXAMPLE 10

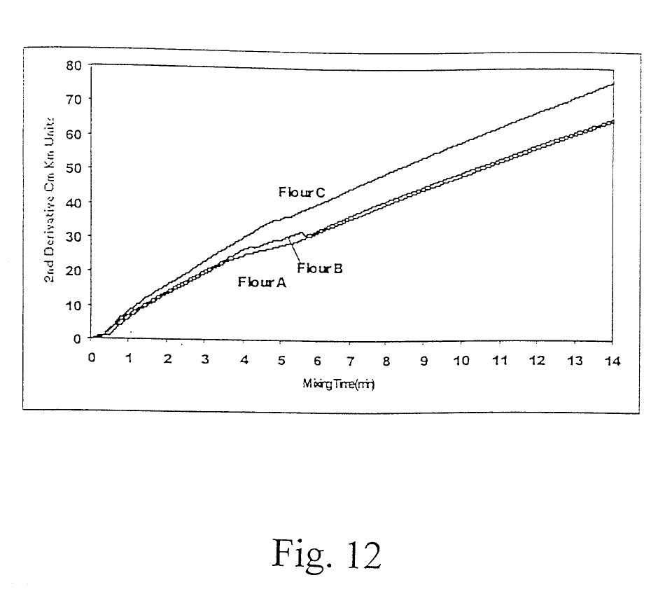

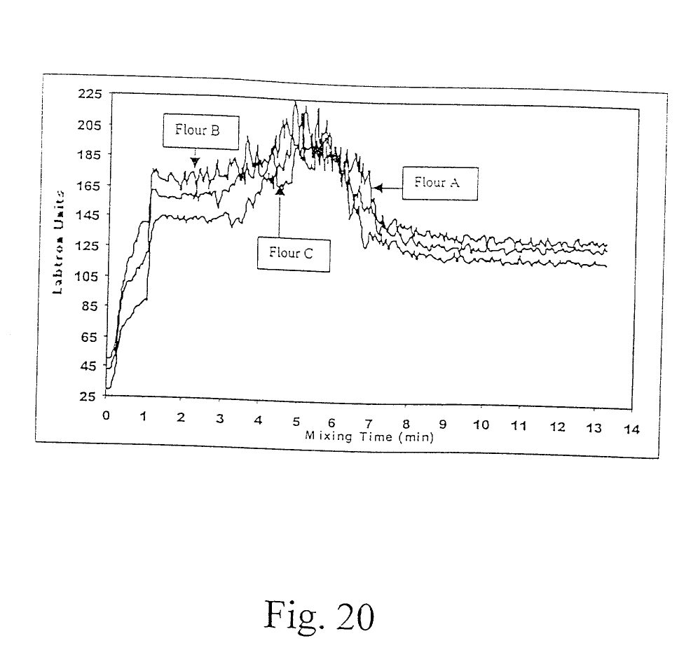

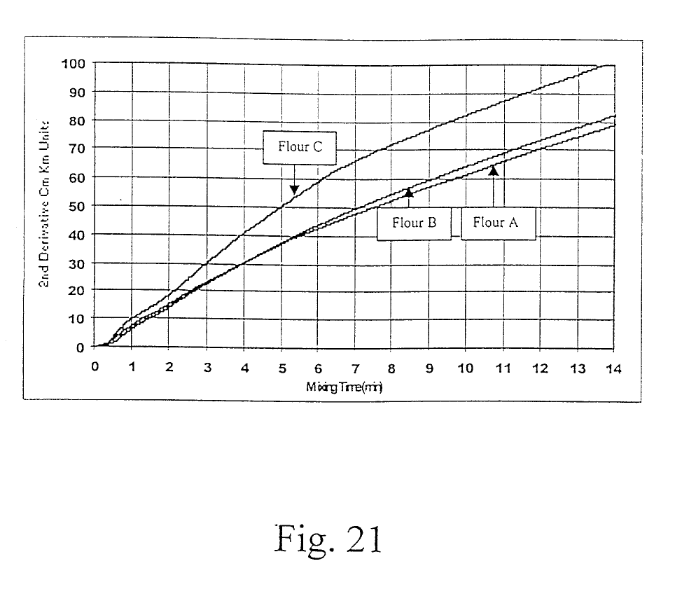

This example demonstrates that differences between flours in otherwise identical doughs can be detected using NIR spectrometry coupled with the data processing as described above. Three doughs were prepared, each including 1100 g of one of three respective Hard Red Winter Wheat flour varieties (flour A, flour B, or flour C), 77 g sugar, 33 g shortening, 22 g compressed yeast at either 56%, 60%, or 64% water absorption level. Each respective flour was tested at each respective water absorption level. FIG. 12 and FIG. 21, which are representative of 60% and 64% water absorption respectively for each flour, show that doughs containing flour A and doughs containing flour B were similar to each other while the dough containing flour C was much different. Confirmation of these differences was shown by Labtron testing at the 60% water absorption level. These results are given in FIG. 20.

The Labtron curve of flour A was different from that of flour B. These curves were different despite the fact that flours A and B are replicates of the same flour.

Analysis of the NIR development plots demonstrated that the optimum water absorption and mixing times were 61% and 5.9 minutes for doughs containing flour A, 61.5% and 5.7 minutes for doughs containing flour B, and 60.5% and 5.3 minutes for doughs containing flour C. It is apparent that the graph generated by NIR spectrometry and processed using the preferred methods as described above allows for much easier identification of flour differences than the graph generated by Labtron testing. The present invention may therefore be used to identify and control the consistency of the quality of flour shipped from millers to bakers.

### EXAMPLE 11

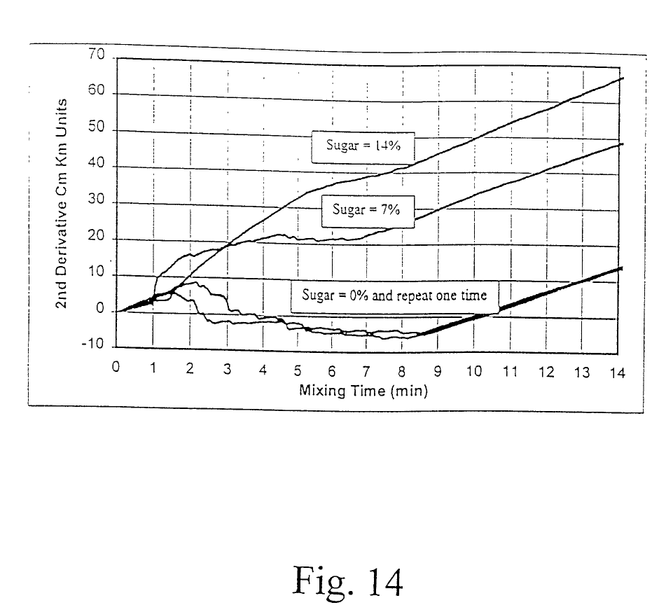

This example demonstrates that the level of sucrose in a dough affects dough characteristics and mixing times using the preferred NIR techniques hereof. Three full formula doughs were formed and each dough included 1100 g Hard Red Winter Wheat flour, 33 g shortening, 22 g compressed yeast, 22 g salt, a 58% level of water absorption and either 0% sugar, 7% sugar or 14% sugar. The experiment using dough having 0% sugar was repeated one time. Results from this testing are given in FIG. 14. Data were collected and processed using the NIR apparatus and data processing as previously described. As shown in FIG. 14, the development curves were greatly effected by the amount of sugar added to each dough. This demonstrates that mixing times and development phases can be predicted based on the amount of sugar added to the dough system. The dough with 7% sugar had a shorter hydration time and dough development time that the dough with 14% sugar. The resistance of dough with a higher sugar level was lower than that of dough with a low sugar level. The sugar in the dough competed with the gluten for the water available in the dough system. This is why the gluten took a longer time for hydration. As a result of the increased hydration time, the dough development time was increased.

### EXAMPLE 12

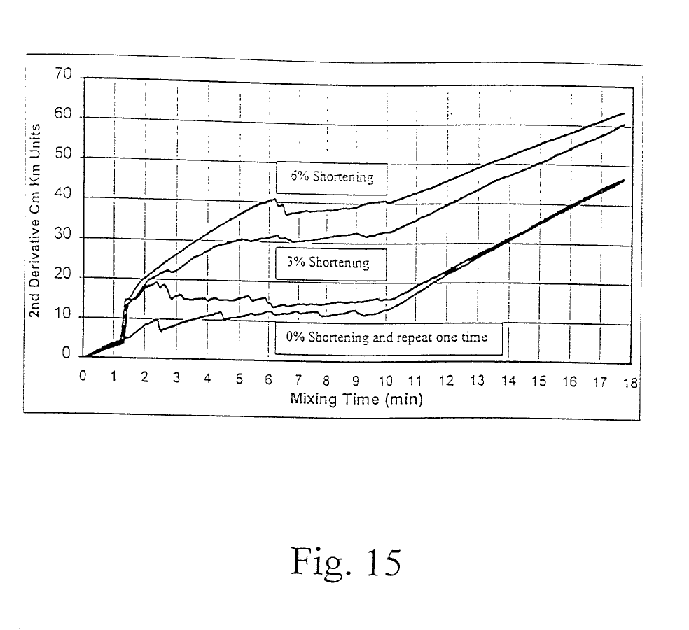

This example demonstrates that the amount of shortening in a dough has an effect on dough characteristics and thereby the dough development curve. Three full formula doughs were formed, each having 1100 g Hard Red Winter Wheat flour of the Karl variety, 77 g sugar, 22 g compressed yeast, 22 g salt, a 58% level of water absorption and either 0% shortening, 3% shortening or 6% shortening. Results of this testing are given in FIG. 15. Once again, data was collected and processed using the NIR apparatus and data processing as previously described. The final graph consists of the second derivative cumulative KM units graphed versus mixing time. It is apparent that the amount of shortening in a dough affects dough characteristics and thereby the dough development curve. After the shortening was added into the dough, dough resistance was decreased with increased shortening. The dough hydration and development time for both the 3% and the 6% shortening levels were not much different. The development plot showed a good repeat. Therefore, NIR can be used to predict dough characteristics based on the amount of shortening in the dough.

### EXAMPLE 13

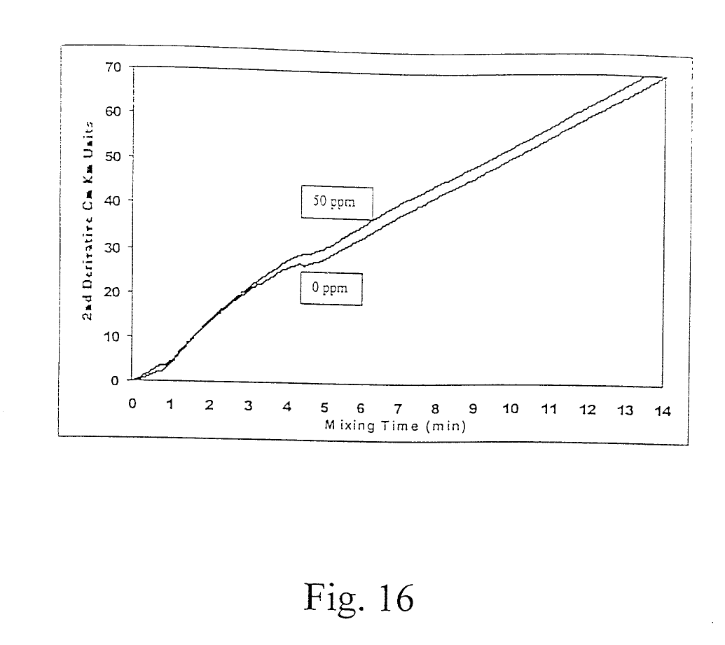

This example demonstrates the effect of potassium bromate on dough characteristics and the dough development curve in full formula doughs. Two full formula doughs were formed, each having 1100 g commercial bread flour, 77 g sugar, 33 g shortening, 22 g compressed yeast, 22 g salt, a 64% water absorption level and either 0 ppm potassium bromate or 50 ppm potassium bromate. Data was collected and processed using NIR spectrometry and data processing as previously described. Results from this testing are given in FIG. 16. When comparing FIG. 16 with FIGS. 3, 8, 9, 11, 12, 14 and 15, it is easy to see that potassium bromate does not greatly modify dough characteristics and the dough development curve. The potassium bromate is a slow acting oxidant and exerts its main function at the oven stage. Thus, dough properties with different potassium bromate levels were not much different during the dough mixing stage as the addition of potassium bromate only slightly increased mixing times. This shows that all oxidants do not have an appreciable effect on mixing time.

### EXAMPLE 14

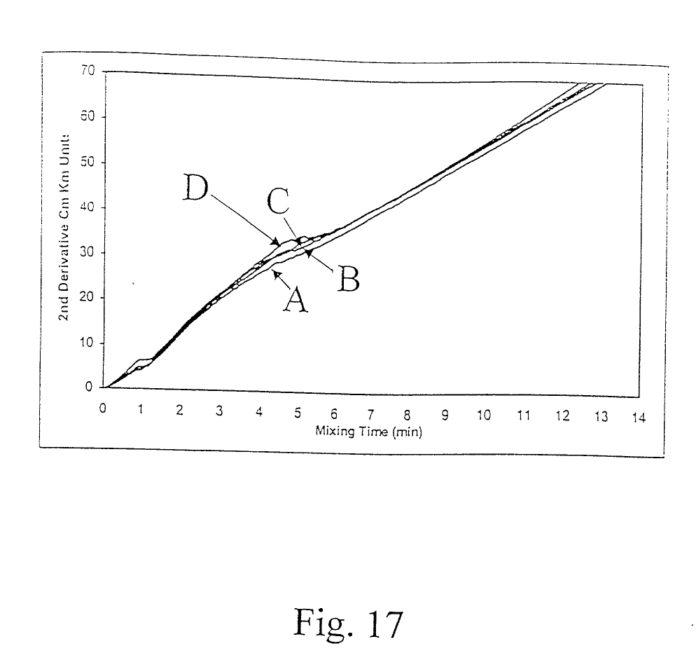

This example demonstrates that dough mixing times and dough characteristics can be affected by the addition of a common oxidant, azodicarbonamide (ADA). Four doughs were formed, each having 1100 g Hard Red Winter Wheat flour, 77 g sugar, 33 g shortening, 22 g compressed yeast, 22 g salt, a 62% level of water absorption and either no ADA (line A), 15 ppm ADA (line B), 30 ppm ADA (line C) or 45 ppm ADA (line D). Data was collected and processed using the preferred NIR apparatus and data processing methods previously described. Results from this testing are given in FIG. 17. The addition of any level of ADA to doughs increased dough hydration time. As the levels of added ADA increased, there was a corresponding increase in mixing times. The differences among the different levels of added ADA were noticeable when using the preferred techniques of the present invention. Based on these results, the present invention maybe used to predict dough characteristics based on the presence or addition of ADA.

### EXAMPLE 15

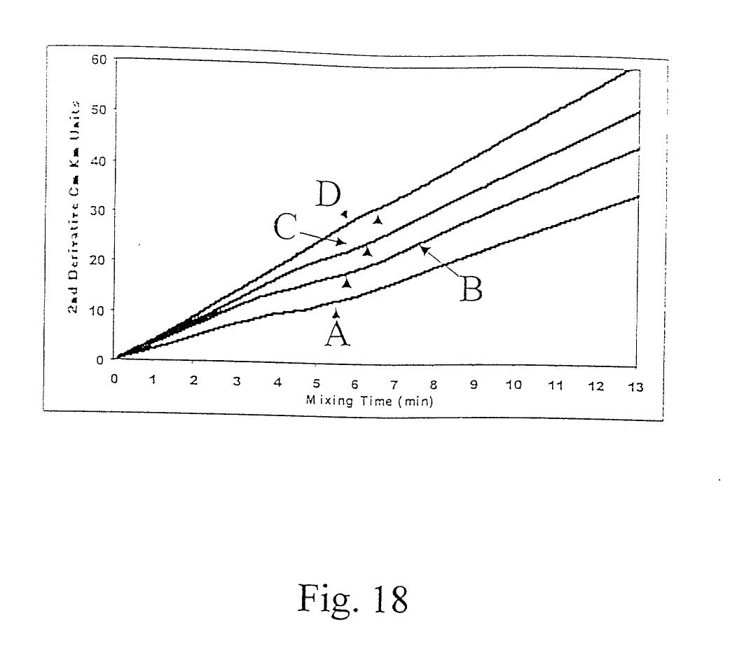

This example demonstrates that a wheat flour sample having different levels of water absorption ranging from 52-58% present different development curves. Four doughs were prepared, each having 1100 g Hard Red Winter Wheat flour (New Crop 99), 77 g sugar, 33 g shortening, 22 g compressed yeast, 22 g salt and either a 52%, 54%, 56% or 58% level of water absorption. Data was collected and processed using the preferred method described above and the results are given in FIG. 18.

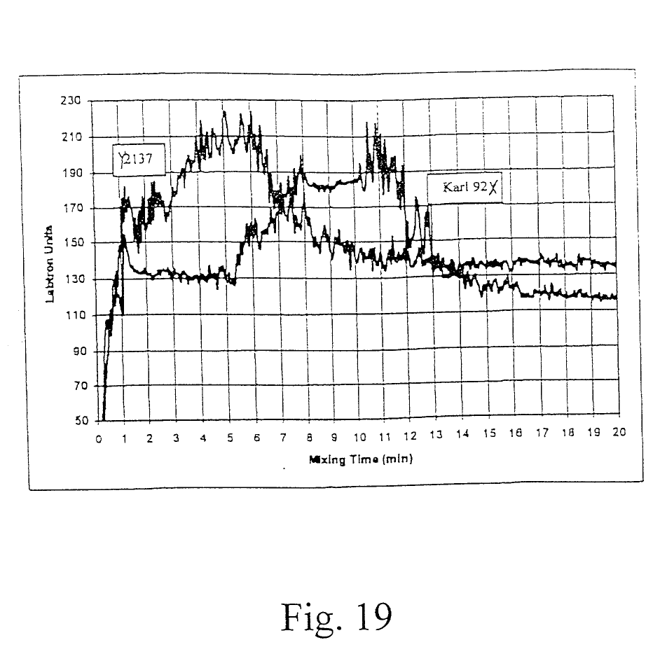

It is apparent that the level of water absorption has a profound effect on dough characteristics and dough development curves. A mark on each line was made to indicate dough mixing time (FIG. 19, A=52%, B=54%, C=56%, and D=58%). The dough mixing time was increased with increasing water levels. The dough hydration time increased with water levels as well.

### EXAMPLE 16

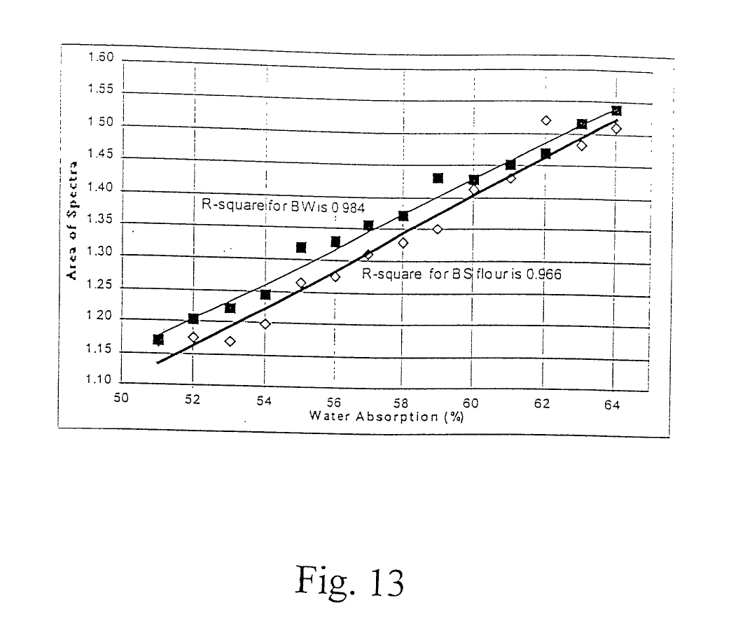

This example demonstrates that flour quality may be determined by NIR based on the slope of areas under wavelengths at different water levels and water absorption rates. Two different commercial bread flours were tested in order to determine the relationship between water levels and the area under the wavelengths from 800 nm to 1600 nm. Testing was performed using two full formula doughs. Each dough included 1100 g flour. The first formula (BW) used a relatively weak flour while the second formula (BS) used a relatively strong flour. Each dough also included 77 g sugar, 33 g shortening and 22 g compressed yeast, and 22 g salt. The raw spectra were processed using a Savitsky-Golay second derivative with 11 point second degree polynomial smoothing. The derivatives were then averaged. The wavelengths included in the testing were from 800 nm to 1675 nm at 5 nm intervals. Each flour had 14 levels of water absorption and most wavelengths from 800 nm to 1600 nm were shown to be highly correlated to these levels of water absorption. There was also a high correlation coefficient (R²>0.95) for each flour between water absorption and area under wavelengths from 800 nm to 1600 nm. Results of this testing are given in FIG. 13.

Each flour may have its own regression line plotted as the different water absorption level versus area of wavelengths (Y=aX+b). The differences among the regression lines for different flours resulted from differences in flour quality. Thus, flour quality could be determined using NIR spectrometry.

### EXAMPLE 17

This example demonstrates that some wavelengths were highly correlated to sucrose. Two commercial bread flours were combined with ten levels of sucrose ranging from 0% to 18% in full formula doughs. These full formula doughs included 1100 g Hard Red Winter Wheat flour, 33 g shortening, 22 g compressed yeast, 22 g salt and a 58% water absorption level. Testing was performed using the preferred NIR methods and data processing as described above. It was found that the wavelengths between 945-955 nm, 995-1005 nm, 1140-1145 nm, 1180-1205 nm, 1330-1340 nm, 1385-1395 nm, 1425-1495 nm, 1575-1580 nm and 1640-1670 nm were highly correlated (R²>0.95) to sucrose.

As shown by examples 16 and 17, certain NIR wavelengths are highly related to specific ingredients commonly found in bread doughs. Knowledge of which wavelengths correlate to specific ingredients allows for monitoring levels of specific ingredients in doughs using specific wavelengths in NIR spectrometry.

### EXAMPLE 18

This example demonstrates that some wavelengths are highly related to the presence of shortening or levels thereof. Two commercial flours were each tested at 13 different levels of shortening ranging between 0% to 12% in full formula doughs. These full formula doughs included 1100 g Hard Red Winter Wheat flour, 22 g compressed yeast, 22 g salt and a 58% water absorption level. Testing was performed using the preferred NIR methods and data processing as described above. It was found that the wavelengths between 890-895 nm,920-930 nm, 1030-1040 nm, 1200-1255 nm, 1340-1350 nm and the wavelength at 955 nm were related to the level of shortening contained in the dough.

### EXAMPLE 19

This example demonstrates that some wavelengths are related to the presence of salt or levels thereof. Two commercial flours were each tested at 9 different levels of salt ranging between 0% to 4% in full formula doughs. These full formula doughs included 1100 g Hard Red Winter Wheat flour, 33 g shortening, 22 g compressed yeast, and a 58% water absorption level. Testing was performed using the preferred NIR methods and data processing as described above. It was found that the wavelengths between 1025-1035 nm and the wavelength at 850 nm were related to the presence and level of salt contained in the product.

We claim:

1. A method of analyzing wheat-based dough or batter products, comprising the steps of:
mixing a plurality of individual dough or batter products, each product including respective quantities of wheat flour, water, and at least one added ingredient selected from the group consisting of shortening, sugar, salt, oxidants, yeast, reducing agents, emulsifiers, and dough improvers, said individual dough or batter products differing from each other in the amount or type of said added ingredient therein;
performing near infrared analyses of each of said products at different times during said mixing step; and
comparing said analyses as an indication of the effect of the amount or type of said added ingredient in said products.

2. The method of claim 1, each of said near infrared analyses being carried out at the same wavelength.

3. The method of claim 2, said wavelength being 1380 nm.

4. The method of claim 1, each of said products being mixed in a bowl, said analyses being performed by directing near infrared radiation into said bowl and measuring an effect of said radiation on said products.

5. The method of claim 4, including the step of measuring the absorbance of said near infrared radiation by said products.

6. The method of claim 1, including the step of generating a mixing time plot for each of said products using data derived from each of said analyses at a specific near infrared wavelength.

7. The method of claim 6, including the step of generating said mixing time plots by determining the second derivative of the spectral data derived from the analyses of each of said products, and graphing such second derivatives versus mixing time.

8. The method of claim 7, each of said plots being generated by first converting said spectral data derived from the analyses of each of said products to Kubelka-Munk units, thereafter determining the second derivative of the Kubelka-Munk converted spectral data, and graphing such second derivatives versus mixing time.

9. The method of claim 8, including the step of cumulating said Kubelka-Munk units for each of said products after determining the second derivatives thereof.

10. The method of claim 1, wherein the amount of said added ingredient is varied in said plurality of products.

11. The method of claim 1, wherein the type of said added ingredient is varied in said plurality of products.

12. The method of claim 1, said oxidant being selected from the group consisting of potassium bromate, ascorbic acid and azodicarbonamide, said reducing agent being selected from the group consisting of L-cysteine, proteases and sodium bisulfite, and said emulsifiers being selected from the group consisting of monoglycerides, diglycerides, combinations of mono and diglycerides, sodium stearoyl lactylates, calcium stearoyl lactylates, diacetyltartaric acid and esters of monoglycerides.

13. A method of analyzing a wheat-based dough or batter product, comprising the steps of:
mixing the ingredients making up said dough or batter product including respective quantities of wheat flour, water, and at least one added ingredient selected from the group consisting of shortening, sugar, salt, oxidants, yeast, reducing agents, emulsifiers, and dough improvers; and
performing near infrared analyses of said product at different times during said mixing step, said analyses being carried out at a wavelength correlated with one of said added ingredients for determining the effect of said added ingredient in said product.

14. The method of claim 13, including the step of performing said near infrared analyses of said product at a plurality of near infrared wavelengths, at least certain of said wavelengths being correlated with corresponding ones of said added ingredients in order to determine the effect of said added ingredients in said product.

15. The method of claim 13, all of said different wavelengths being correlated with corresponding ones of said added ingredients.

16. The method of claim 13, said product being mixed in a bowl, said analyses being performed by directing near infrared radiation into said bowl and measuring an effect of said radiation on said product.

17. The method of claim 15, including the step of measuring the absorbance of said near infrared radiation by said product.

18. The method of claim 13, including the step of generating a mixing time plot for said product using data derived from said analyses.

19. The method of claim 18, including the step of generating said mixing time plots by determining the second derivative of the spectral data derived from the analyses of said product, and graphing such second derivative versus mixing time.

20. The method of claim 19, said plot being generated by first converting said spectral data derived from the analyses of said product to Kubelka-Munk units, thereafter determining the second derivative of the Kubelka-Munk converted spectral data, and graphing such second derivative versus mixing time.

21. The method of claim 20, including the step of first cumulating said Kubelka-Munk units for said product prior to determining the second derivative thereof.

22. The method of claim 13, said oxidant being selected from the group consisting of potassium bromate, ascorbic acid and azodicarbonamide, said reducing agent being selected from the group consisting of L-cysteine, proteases and sodium bisulfite, and said emulsifiers being selected from the group consisting of monoglycerides, diglycerides, combinations of mono and diglycerides, sodium stearoyl lactylates, calcium stearoyl lactylates, diacetyltartaric acid and esters of monoglycerides.

23. The method of claim 13, said product being a dough.

24. The method of claim 23, said dough being yeast-leavened.

25. The method of claim 23, said dough being chemically leavened.

26. The method of claim 13, said product being a batter.
